(19) United States
(12) Patent Application Publication
(10) Pub. No.: US 2012/0055485 A1
(43) Pub. Date: Mar. 8, 2012

(54) WEARABLE MEDICAL SUPPORT FOR DELIVERY OF FLUIDS TO THE NOSE

(76) Inventor: Jean-Michel Anthony, Kapellen (BE)

(21) Appl. No.: 13/266,739

(22) PCT Filed: Apr. 27, 2010
(86) PCT No.: PCT/EP10/55643

§ 371 Date: Oct. 27, 2011

(30) Foreign Application Priority Data
Apr. 27, 2009 (EP) 09158849.1

(51) Int. Cl. A61M 16/06; A61M 39/12

(52) U.S. Cl. 128/207.18; 604/94.01; 604/174

(57) ABSTRACT

A wearable medical support is provided for delivery of fluids to the nose of a subject. The support includes a heat moldable nose mask having a longitudinal direction, formed from a sheet of thermoplastic material. The nose mask includes a nose aperture dimensioned to fit the nose of the subject, and a fixture for a strap at each opposing longitudinal end of the mask The support includes a coupling dismountably fixed to the mask for attachment of one or more tubes for delivery of the fluid to the nose. The nose mask is configured for individual molding across at least part of the cheek bones of the subject.

### FIELD OF THE INVENTION

The present invention is in the field of a wearable support for the delivery of fluids through the nose of a subject. In particular, it concerns a support that can be worn for the treatment of sleep apnea, or for supporting a catheter providing air to the lungs or liquid nutrition to the stomach.

### BACKGROUND TO THE INVENTION

A variety of supports are known in the art that may be worn by a subject and which secure a tube for the delivery of a fluid (e.g. a liquid, a gas) through the nose.

The most common supports are respiratory masks, which are triangular in shape to compliment the nose, and seal against the skin of the subject, as described for instance, in U.S. 2006/0096598. A disadvantage of such masks is the weight and the low level of comfort they afford the wearer. Because they seal using a triangular facial component, pressure is applied to the skin in a concentrated region along the triangle edges. They result in unseemly pressure marks to the skin of the wearer after use, which marks are not only attributable to the profile of the mask, but also to the accompanying straps which pass across the face of the wearer. Such masks may be worn only for limited period of time without the risk of adverse reaction. They are unsuitable for wearing at night, as required by sufferers of sleep apnea for long and repeated periods.

An alternative type of support is for a catheter tubing which is employed for the delivery of fluids such as gas or liquid to the nose. Examples include breathing and feeding tubes which are passed down the wind pipe and esophagus respectively. Such tubes are commonly held in placing using an adhesive strip applied to the skin of the face. However, the strips are not strongly adhesive against the friction-resistive tube walls and so are prone to dislodging when the patient moves, thereby risking the possibility that catheter is displaced. Moreover, adhesive strips commonly elicit an allergic reaction in the wearer. Because the strips are worn on the face, the appearance of an allergic rash is highly undesirable. Moreover, allergic rashes are known to be permanent or reoccur for months or years after the event.

In view of the problems of the art, the present invention aims to provide a new support avoids the problems of the art.

### SUMMARY OF THE INVENTION

The present invention is related to an wearable medical support (100) for delivery of fluids to the nose of a subject comprising:
a heat moldable nose mask (10), having a longitudinal direction, formed from a sheet of thermoplastic material configured for individual molding across at least part of the cheek bones of the subject, comprising:
   a nose aperture (12) dimensioned to fit the nose of the subject, and
   a fixture (14, 16) for a strap at each opposing longitudinal end of the mask (10), and
a coupling (20, 20a 40, 45) fixed to the mask (10) for attachment of one or more tubes for delivery of the fluid to the nose. The coupling may be permanently or dismountably fixed to the mask (10).

Another embodiment of the invention is the support (100) as described above, where the coupling is dismountable, and is comprised in a hollow tubular fitting (20a to c) having a proximal (22) and distal (24) end,
the proximal end (22) of the tubular fitting (20a to c) being provided with a flanged (28) opening (82) for receiving the nose,
the distal end (24) being provided with the inlet port (21) for attachment to a tube for delivery of the fluid to the nose, and
a passageway (26) connecting the inlet port (21a to c) to the flanged opening (82).

Another embodiment of the invention is the support (100) as described above, wherein the flange (28) is provided on a skin-facing side (32) of the nose mask (10), the inlet port (21) is provided on an exterior side (30) of the nose mask (10), and the passageway (26) is disposed through the nose aperture (12), so mounting the mask (10) over the passageway (26), and in abutting alignment with the proximal (22) side of the flange (28).

Another embodiment of the invention is the support (100) as described above, wherein the outer profile of the passageway (26) is essentially triangular, or essentially isosceles trapezoidal.

Another embodiment of the invention is the support (100) as described above, wherein the inlet port (21b, c, d) is circular, essentially triangular, or essentially isosceles trapezoidal.

Another embodiment of the invention is the support (100) as described above, wherein the inlet port (21b) and passageway (26) are configured to pass slidably through the nose aperture (12).

Another embodiment of the invention is the support (100) as described above, wherein the passageway (26) is at least partially bellowed.

Another embodiment of the invention is the support (100) as described above, wherein the flange (28) is configured to pass through the nose aperture (12) in a folded state.

Another embodiment of the invention is the support (100) as described above, wherein the nose aperture (12) is essentially triangular or isosceles trapezoidal, the base of the triangle or isosceles trapezium oriented in the longitudinal direction of the nose mask (10).

Another embodiment of the invention is the support (100) as described above, wherein the nose aperture (12) is essentially triangular, the base of the triangle oriented in the longitudinal direction of the nose mask (10).

Another embodiment of the invention is the support (100) as described above, wherein the longitudinal width (W) of the mask (10) is between 16 cm and 22 cm.

Another embodiment of the invention is the support (100) as described above, wherein the longitudinal width (W) of the mask (10) is larger than that of the lateral height (H) of the mask, preferably between 1.5 and 3 times larger.

Another embodiment of the invention is the support (100) as described above, wherein the nose mask (10) comprises a thermoplastic composition containing polycaprolactone and polyurethane.

Another embodiment of the invention is the support (100) as described above, wherein the nose mask (10) comprises is disposed with a plurality of aeration apertures.

Another embodiment of the invention is the support (100) as described above, wherein the aeration apertures are provided at a density of between 0.5 and 5 apertures per cm².

Another embodiment of the invention is the support (100) as described above, wherein the nose mask (10) is made substantially from a sheet material comprising:
a core layer (60) having an upper surface (50) and lower surface (52), that is a thermoplastic composition comprising polycaprolactone and polyurethane.

Another embodiment of the invention is the support (100) as described above, further comprising a first outer layer (55) disposed over the upper surface of the core layer that is a body-side liner, which layers are bonded so as to form a single sheet.

Another embodiment of the invention is the support (100) as described above, further comprising a second outer layer (65) disposed over the lower surface of the core layer comprising polyurethane, polyester polyurethane or polyether open-cell foam, which layers are bonded so as to form a single sheet

Another embodiment of the invention is the support (100) as described above, wherein the core layer (60) comprises 20% to 40%, polyurethane, and 60% to 80% (w/w) polycaprolactone.

Another embodiment of the invention is the support (100) as described above, wherein the material of the first outer layer (55) is formed from
a yarn comprising polyamide and elastane, preferably comprising between 80% to 95% polyamide, and between 5% and 15% elastane,
   elasticated neoprene, or
   flock.

Another embodiment of the invention is the support (100) as described above, further comprising an intervening layer (60, 65) disposed between the core layer (60) and the first outer layer (55), and/or disposed between the core layer (60) and the second outer layer (65), made from the same material as the core (60) and with a higher polycaprolactone content.

Another embodiment of the invention is the support (100) as described above, wherein the mask (10) material has a maximum total thickness of 1.5 to 1.7 mm.

Another embodiment of the invention is the support (100) as described above, wherein the coupling is comprised in an essentially triangular tube (20—FIGS. 1 to 3), sealed over the nose aperture (12) at one end, and at the other end provided with a port for attachment to a fitting for providing gas. The coupling is preferably permanently fixed. The tube may be rubberized or made from silicone.

Another embodiment of the invention is the support (100) as described above, wherein the coupling is comprised in an adapter (45—FIGS. 14 to 16) having a pair of adjustable gas outlet nozzles (46, 46′) in fluid connection with an inlet port (48) for coupling to a fitting for providing gas.

Another embodiment of the invention is the support (100) as described above, wherein the coupling is comprised in a tube clip (40—FIGS. 17 to 19) configured to secure a fluid delivery tube, such as a catheter, to the nose mask and align it with the entrance to a nostril.

Another embodiment of the invention is a dismountable coupling as defined above. The flange (28) may have a profile corresponding at least partially to that of the nose mask (10) as defined above

### FIGURE LEGENDS

FIG. 1 depicts a support of the invention disposed with a dismountable or permanently fixed coupling that is a hollow tubular fitting for attachment to a supply of air, whereby the nose mask has a curved profile prior to molding.

FIG. 2 depicts a support of the invention as shown in FIG. 1, after molding to fit the contours of the region around the nose. The view is of the front of the mask.

FIG. 3 depicts a support of the invention as shown in FIG. 1, after molding to fit the contours of the region around the nose. The view is of the back (skin contacting or facing) side of the mask.

FIG. 4 depicts the support of FIG. 1, highlighting a longitudinal (central) axis A-A′ of the coupling between its proximal and distal ends.

FIGS. 5A to 5D depict different views of a dismountable coupling that is a hollow tubular fitting provided with a proximal flexible flange: FIG. 5A is a view with the proximal end facing the viewer, FIG. 5B is a view with the distal end facing the viewer, FIG. 5C is a cross-sectional view perpendicular to the longitudinal (A-A′) axis, FIG. 5D is a perspective view.

FIG. 6 is a cross-sectional view of the coupling perpendicular to the longitudinal, mounted in the aperture of the nose mask 10.

FIG. 7 depicts a removable coupling of the invention attached to the support. The view is of the back (skin contacting or facing) side of the mask. The dotted line 84 indicates that the flange can extend to the periphery of the mask.

FIG. 8 depicts an embodiment of the flange part of a coupling embodiment with indicated dimensions.

FIGS. 9A to 9E depict views of a dismountable coupling that is a hollow tubular fitting provided with a proximal flexible flange: FIG. 9A is a view with the proximal end facing the viewer, FIG. 9B1 is a plan view of the coupling provided with a planar flange, FIG. 9B2 is a plan view of the coupling provided with a curved flange, FIG. 9C is a perspective view. FIG. 9D is a perspective view of the coupling mounted on the mask, FIG. 9E is a cross-sectional view perpendicular to the longitudinal (A-A′) axis.

FIGS. 10A to 10E depict views of a dismountable coupling that is a hollow tubular fitting provided with a proximal flexible flange: FIG. 10A is a view with the proximal end facing the viewer, FIG. 10B is a plan view; FIG. 10C is a perspective view. FIG. 10D is a perspective view of the coupling mounted on the mask; FIG. 10E is a cross-sectional view perpendicular to the longitudinal (A-A′) axis.

FIGS. 11A to 11C depict views of a dismountable coupling that is a hollow tubular fitting provided with a proximal flexible flange and a bellowed region of the passageway: FIG. 11A is a view with the proximal end facing the viewer; FIG. 11B is a plan view; FIG. 11C is a perspective view.

FIG. 12 depicts a nose mask of the invention adapted to slidably receive the dismountable coupling of FIGS. 9A to 9E, 10A to 10E and FIGS. 11A to 11C.

FIG. 13 depicts an alternative embodiment of the flange part of a coupling embodiment with indicated dimensions.

FIG. 14 depicts a support of the invention disposed with a nozzled coupling for attachment to a supply of air, whereby the nose mask has a curved profile prior to molding.

FIG. 15 depicts a support of the invention as shown in FIG. 14, after molding to fit the contours of the region around the nose. The view is of the front of the mask.

FIG. 16 depicts a support of the invention as shown in FIG. 14, after molding to fit the contours of the region around the nose. The view is of the back (skin contacting or facing) side of the mask.

FIG. 17 depicts a support of the invention suitable for coupling to fluid catheter for insertion into the nose, whereby the nose mask has a curved profile prior to molding.

FIG. 18 depicts a support of the invention as shown in FIG. 17, after molding to fit the contours of the region around the nose. The view is of the front of the mask.

FIG. 19 depicts a support of the invention as shown in FIG. 17, after molding to fit the contours of the region around the nose. The view is of the back (skin contacting or facing) side of the mask.

FIG. 20A and 20B depicts two alternative embodiments of the mask element of a support of the invention with indicated dimensions.

FIG. 21 depicts a schematic drawing of a cross section through a sheet of thermoplastic material used to form the nose mask element of the support comprising a core material.

FIG. 22 depicts a schematic drawing of a cross section through a sheet of thermoplastic material used to form the nose mask element of the support, comprising a core material provided with a first outer (skin contact or facing) layer.

FIG. 23 depicts a schematic drawing of a cross section through a sheet of thermoplastic material used to form the nose mask element of the support, comprising a core material provided with a first outer (skin contact or facing) layer and a second outer (exterior) layer.

FIG. 24 depicts a schematic drawing of a cross section through a sheet of thermoplastic material used to form the nose mask element of the support of FIG. 23, further comprising intervening layers.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “a fixture” means one fixture or more than one fixture.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of fixtures, and can also include 1.5, 2, 2.75 and 3.80, when referring to, for example, measurements). The recitation of end points also includes the end point values themselves (e.g. from 1.0 to 5.0 includes both 1.0 and 5.0)

Reference is made in the description below to the drawings which exemplify particular embodiments of the invention; they are not at all intended to be limiting. The skilled person may adapt the device and substitute components and features according to the common practices of the person skilled in the art.

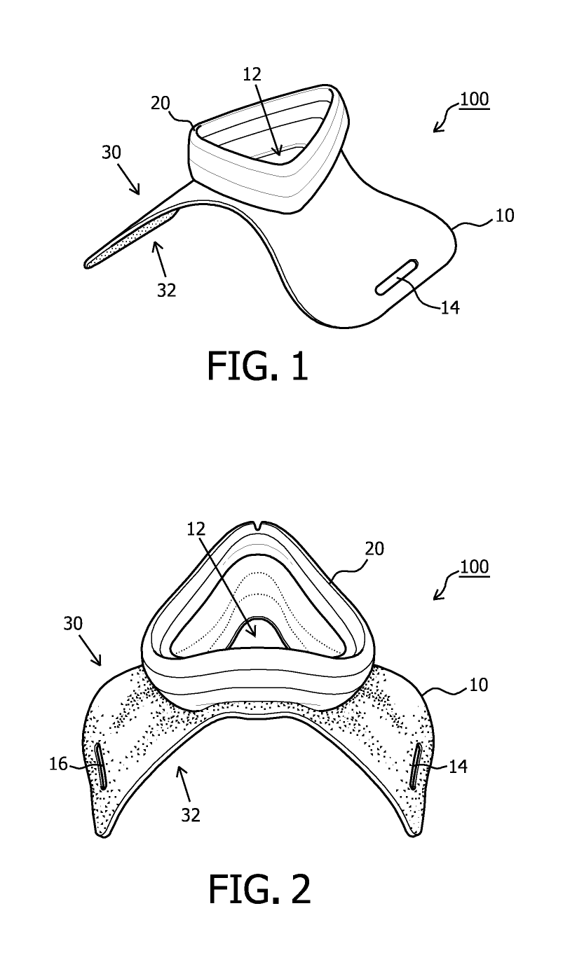

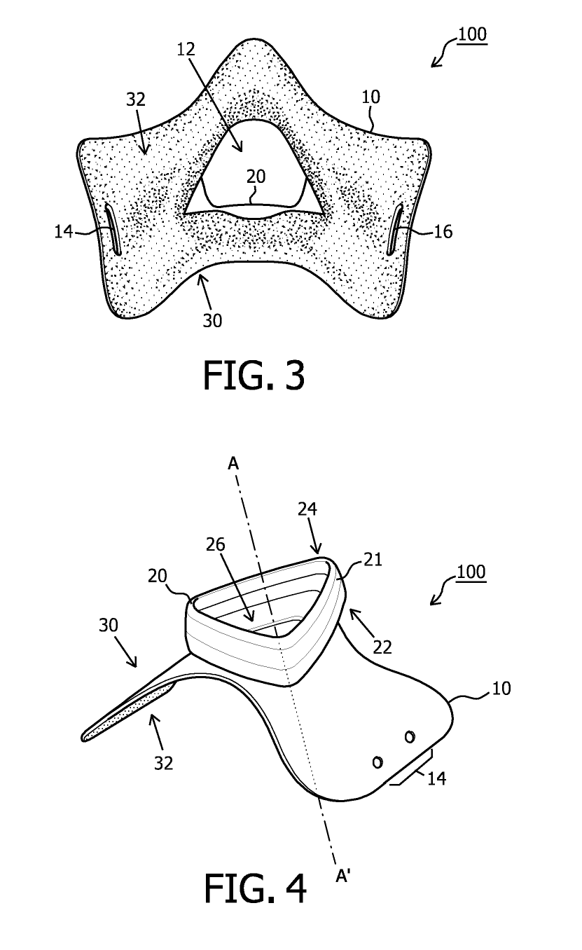

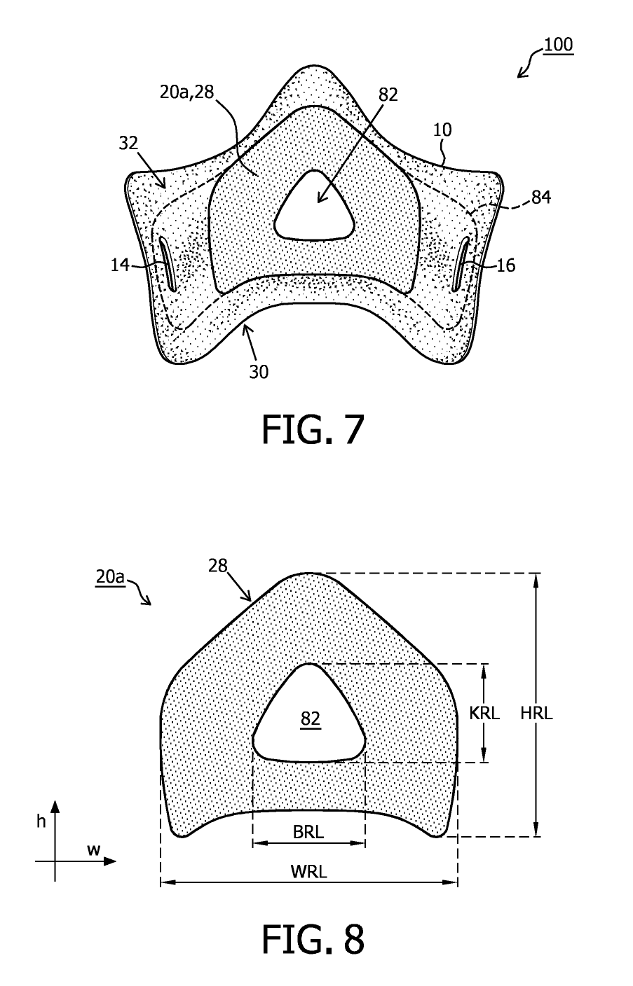

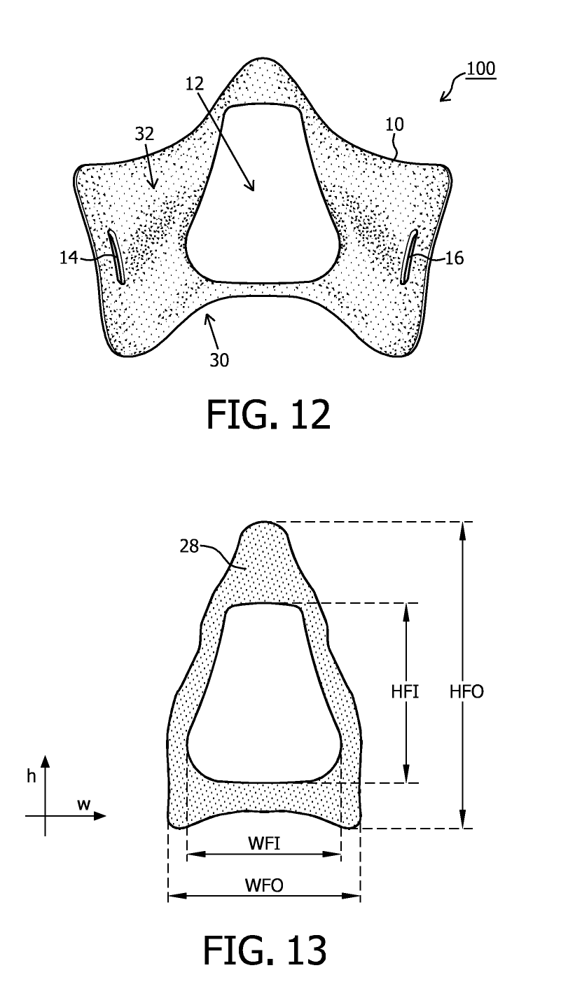

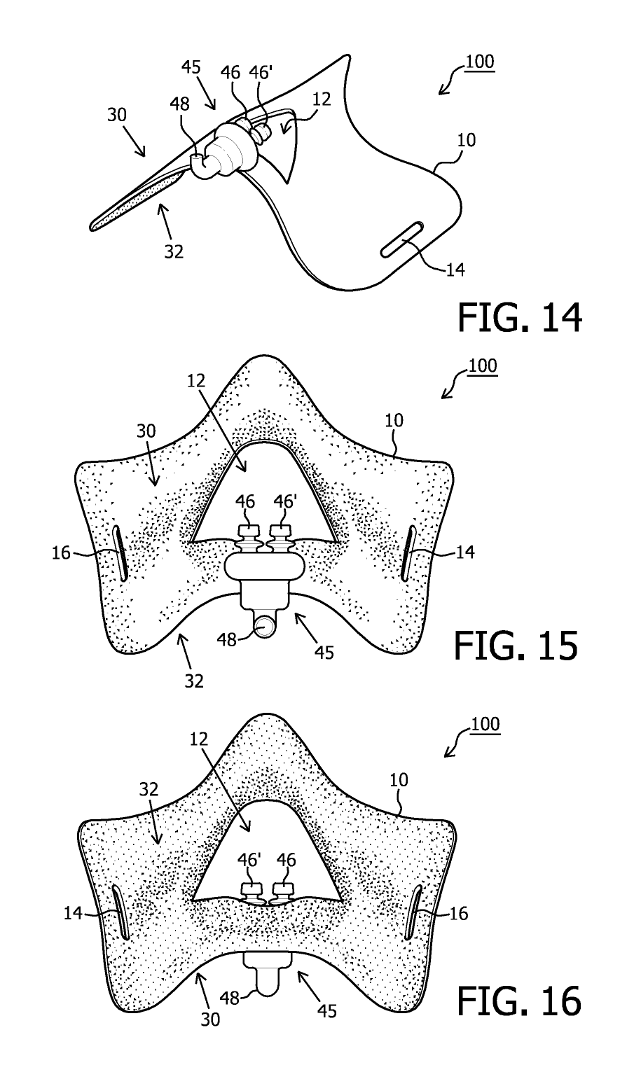

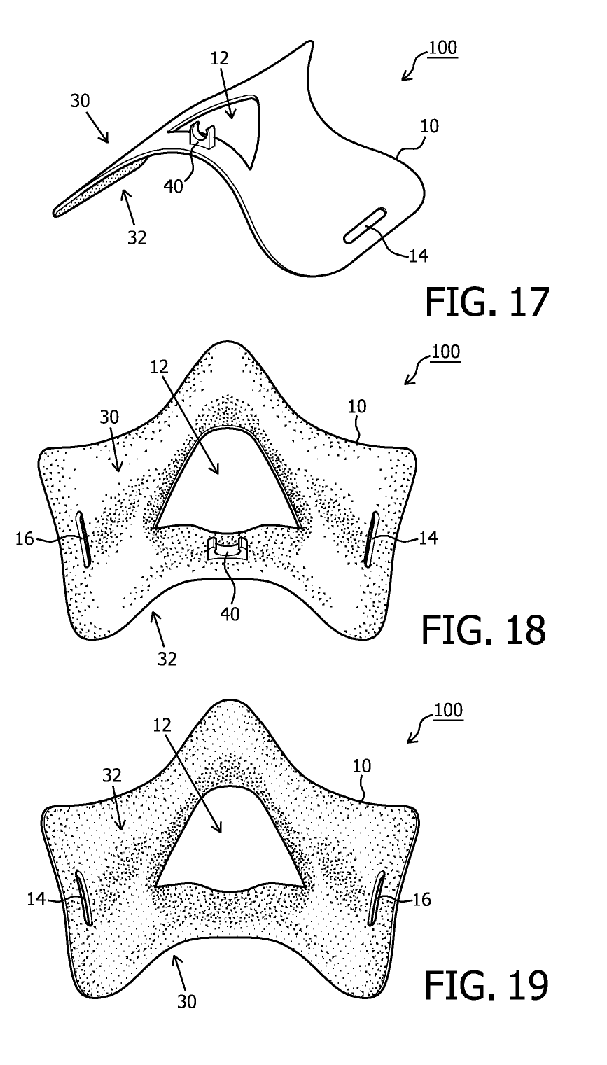

With reference to FIGS. 1 to 19, the present invention concerns a wearable support 100 formed from a heat moldable nose mask 10 comprising an aperture 12 for the nose, fixtures 14, 16 for attachment to a strap, and a coupling 20, 20a, 40, 45 for attachment of one or more tubes for delivery of the fluid to the nose. The coupling in FIGS. 1 to 11C is comprised in a hollow tubular fitting 20, 20a in connection with the aperture 12 adapted to receive a coupling from an air supply; the coupling in FIGS. 14 to 16 is comprised in a dual-nozzled adapter 45 having an inlet port for coupling to an air supply; the coupling in FIGS. 17 to 19 is comprised in a tube clip 40 on the outer surface of the mask 10.

The nose mask 10 element of the support 100 is heat moldable, meaning it may be adapted to the contours of the face so providing a comfort fit enabling it to be worn for extended periods. Where the support 100 is used for delivery of pressurised air, for instance, in the treatment of sleep apnea, the individual fit also provides a partial sealing function. The skin contact or facing surface 32 of the mask 10 may be lined with a softened material such as a felt, neoprene or knitted material. Alternatively, the support 100 may be supplied with a removable layer which provides the softened effect. The use of a softened material not only provides a pleasant feeling against the skin, but also allows a partial flow of circulating air across rather than through the mask, thereby reducing the build up of perspiration and heat below the mask. The mask 10 is particularly suitable for wearing at night without discomfort, and so may be utilised in the treatment of sleep apnea when the fluid delivered to the noise is air. Equally, the support can be used as an attachment point for a feeding tube or breathing tube inserted through the nose. The support 100 is washable.

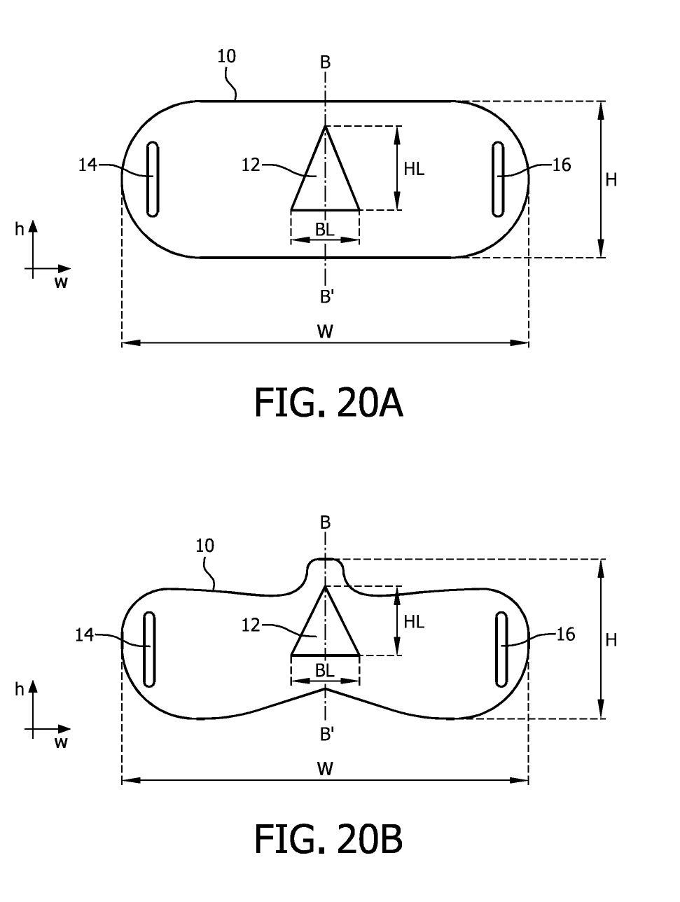

The nose mask 10 element of the support 100 is dimensioned to fit over the nose of the subject. It extends from the nose around at least part of the cheeks, and comprises a pair of fixtures 14, 16 for a strap which passes over the back of the head. The mask 10 is disposed with an aperture 12 for the nose that is preferably essentially triangular in shape, but may alternatively be round, square, oblong or any other suitable shape. It may have the shape substantially of an isosceles trapezium. The shape will be largely determined by the shape of the nose. As will be appreciated, nose mask 10 may be dimensioned so as not to extend over the eyes, mouth or ears. The mask 10 is devoid of apertures for the eyes, mouth or ears. However, an edge of the mask may extend partially around, but not over, the eyes, mouth or ears. For instance, in FIG. 20B the upper edge of the mask 10 has a profile that extends partially around the bridge of the nose between the eyes, but it avoid extending over the eyes.

Prior to molding, the nose mask 10 may be flat, or rounded (e.g. U-shaped) as shown in FIGS. 1, 2, 3, 4, 7, 9D, 10D, 12 and 14 to approximate to the profile of the face. After molding FIGS. 2, 3, 15, 16, 18 and 19 the mask is adapted to match the facial contours of the subject.

The nose mask 10 element of the support 100 has a substantially longitudinal shape. With reference to FIGS. 20A and 20B, the mask 10 has a longitudinal (w) direction, and perpendicular thereto, a lateral direction (h). The nose aperture 12 is preferably centrally located along the longitudinal length of the nose mask 10. The nose aperture 12 may also be centrally located with respect to the lateral height. The nose mask 10 thus comprises two wings which extend from and flank the nose aperture 12. Each wing is adapted to extend from the nose around at least part of the cheeks. Each longitudinal end of the nose mask is disposed with a fixture 14, 16 for a strap. The mask is configured for wearing such that the longitudinal direction of the mask is oriented essentially parallel to the wearer's eye-line or his left-right axis.

The dimension of the longitudinal width (W, FIG. 20A, 20B) of the nose mask 10 may be defined as the surface distance between the extremities of the opposing longitudinal edges of the mask. The distance is measured in the longitudinal (w) direction. The dimensions of the longitudinal width (W) depend on the characteristics of the subject such as age, shape, and gender, but may be equal to or no more than 15 cm, 16 cm, 17 cm, 18 cm, 19 cm, 20 cm, 21 cm, 22 cm, 23 cm, 24 cm, 25 cm or a value in the range between any two of the aforementioned values, preferably between 16 and 22 cm.

The dimension of the lateral height (H, FIG. 20A, 20B) of the nose mask 10 may be defined as the minimum surface distance between the extremities of the opposing lateral edges of the mask. The distance is measured along the lateral (h) direction. The dimension of the lateral height (H) will depend on the characteristics of the subject such as age, shape, and gender, but may be equal to or no more than 7 cm, 8 cm, 9 cm, 10 cm, 11 cm, 12 cm, or a value in the range between any two of the aforementioned values, preferably between 7 and 12 cm.

The dimension of the longitudinal width (W) is larger than that of the lateral height (H) of the mask. Preferably the dimension of the longitudinal width (W) is 1.5, 2, 2.5, 3, 4, 5, or 6 times larger than that of the lateral height (H), or a value in the range between any two of the aforementioned values, preferably between 1.5 and 3 times.

The aperture 12 for the nose is dimensioned such that the nose substantially passes there through. It is of sufficient size that at least the nostrils pass entirely through the aperture, for instance, during molding. It is of sufficient size that at least the nasal tip passes entirely through the aperture, for instance, during molding. The aperture may be substantially, partly or entirely triangular, round, oblong, square of any other suitable shape. It may have the shape substantially of an isosceles trapezium. The shape will be largely determined by the shape of the nose. Preferably, the aperture is substantially triangular in shape, the triangle having a base that corresponds to the nostril region of the nose. The triangle also has an apex that corresponds to the nasal bone. The orientation of the triangle is such that the base lies in the longitudinal direction (w) of the nose mask 10; the triangle base does not lie in the lateral direction (h) of the nose mask 10. Preferably the dimension of the base of the triangle (BL) is 1 cm, 2 cm, 3 m or 4 cm in length. Preferably the perpendicular height of the triangle relative to the base (HL) is 3 cm, 4 cm, or 5 cm in length. Because the aperture allows the nose to pass through, the support avoids that securing pressure is applied to the nose which would otherwise cause discomfort, particularly to the nasal sinuses.

The nose mask 10 may be flat or at least partly curved in the unmolded condition. The above-mentioned dimensions are most preferably obtained when the mask is in the flattened condition, though it will be appreciated that a curved mask can also be measured by transforming a digital model to a flattened state or by measuring along its surface. The curvature may be centered on the aperture 12. As shown in FIGS. 1 and 4 the curvature may be around the central lateral axis B-B′ (FIGS. 20A and 20B). Preferably, the face mask in the lateral direction (h) remains flat in the unmolded condition.

The nose mask 10 is dimensioned to extend from the nose and over the cheek bones. The opposing longitudinal ends of the mask 10 are each provided with a fixture 14, 16 for a strap; because the longitudinal shape positions the strap effectively at the side of the head, and not on the face, the strap does not cut into the face when securing pressure is applied unlike masks of the art. This allows the strap to be worn for prolonged time periods without facial damage, or unwarranted reaction against the strap material. Moreover, the face mask 10 in the molded condition has a large and even area in contact with the surface of the face. Pressure exerted by the strap is evenly distributed, without pressure spots that can lead to an adverse reaction.

The fixtures 14, 16 for the strap may be a pair of slots as depicted, for instance, in FIGS. 1 to 4, 9D, 10D, 12, 14 to 19, however, it is not necessarily limited thereto. The fixtures 14, 16 may comprise one part of a Velcro strip, or one part of a press-stud or snap-fastener, which attaches to a reciprocating element on the strap. Alternatively, each fixture 14, 16 may comprise a pair of holes as indicated, for example, in FIG. 4.

The nose mask 10 may be provided with a plurality of aeration apertures that serve to increase the flow of air to the skin and thereby reduce the build up of perspiration and heat below the mask. The apertures also serve to reduce the weight of the support 100. The aeration apertures may connect the skin contact or facing surface 32 of the mask with the exterior surface 30 i.e. pass from one surface of the mask to the other. Alternatively, the aeration apertures may pass only partially through the mask, for instance through the core and optional intervening layers thermoplastic elements but not through the first outer layer and/or second outer layer (see below). Preferably, the aeration apertures may comprise circular holes; they may have a diameter of 0.5 mm, 1 mm, 1.5 mm, 2.0 mm, 2.5 mm, 3 mm, 3.5 mm, 4 mm, or 5 mm or a value in the range between any two of the aforementioned values; the holes may be present in a region of mask 10 at a density of between 0.5 and 5 holes per cm². The aeration apertures may alternatively or in addition, comprise slots; they may have a length of 1 cm, 2 cm, 3 cm, 4 cm, 5 cm or 6 cm or a value in the range between any two of the aforementioned values, and a width of 0.5 mm, 1 mm, 1.5 mm, 2.0 mm, 2.5 mm, 3 mm, 3.5 mm, 4 mm or 5 mm or a value in the range between any two of the aforementioned values. The aeration apertures may be restricted to the region of the wings.

The nose mask 10 is made from a thermoplastic sheet material 200 deformable under the application of heat to conform to the contours of the face of a subject, which after cooling down retains the deformed shape and becomes rigid or semi-rigid. The nose mask comprises a thermoplastic composition containing polycaprolactone and polyurethane. According to one embodiment, one or both surfaces of the thermoplastic sheet material 200 is unlined. According to one embodiment, at least one surface is lined with a material such a felt, neoprene, or a knitted fabric that provides a comfortable feeling against the skin.

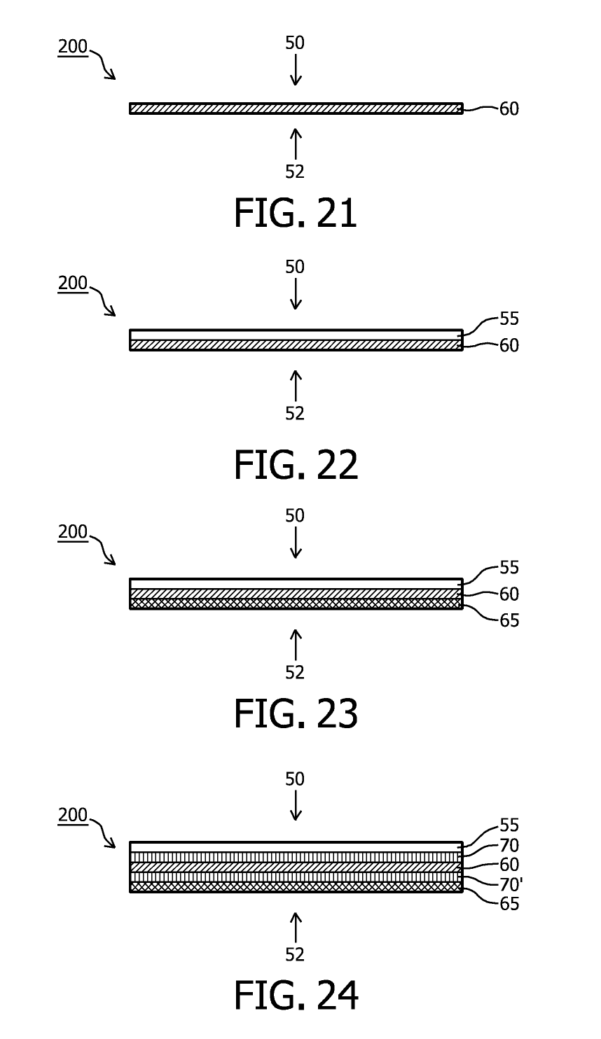

According to one embodiment of the invention, and with reference to FIG. 21, the sheet material 200 comprises a core layer 60 having a first 50 (skin facing 32) side and second 52 (exterior surface 30) side. The core layer 60 may be provided with a first outer layer 55 disposed over the first 50 side surface of the core layer (FIG. 22)—which layers are bonded so as to form a single sheet 200. Alternatively, the first layer may be absent (FIG. 21) in which case the support 100 may be supplied with a removable layer, that is a sheet of soft material applied to the skin facing 32 side of the nose mask 10 and which gives the support 100 a comfortable feeling against the skin. The removable layer has a shape profile essentially the same as that of the mask 10. The removable layer is disposable or washable. It may be formed from a woven or non-woven material. The removable layer may be formed from a material that is gas and vapour permeable. Where it is not, it may be provided with the aforementioned aeration apertures, or with a plurality of perforations to increase breathability. The perforations may have a diameter of 0.1 mm, 0.2 mm, 0.3 mm, 0.4 mm, 0.5 mm, 0.6 mm, 0.7 mm, 0.8 mm, 0.9 mm, or 1 mm or a value in the range between any two of the aforementioned values.

According to another embodiment of the invention as depicted in FIG. 23, the sheet material 200 preferably comprises at least three separate layers—a core layer 60 having a first 50 (skin facing 32) side and second 52 (exterior surface 30) side, a first outer layer 55 disposed over the first 50 side surface of the core layer and a second outer layer 65 disposed over the second 52 side surface of the core layer—which layers are bonded so as to form a single sheet 200.

One or more intervening layers 70, 70′ (FIG. 24) may be disposed between the core layer 60 and the first outer layer 55, and/or disposed between the core layer 60 and the second outer layer 65.

The overall thickness of the sheet 200 may be 1.5 mm, 1.6 mm, 1.7 mm, 1.8 mm, 1.9 mm, 2.0 mm, 2.2 mm, 2.4 mm, 2.5 mm, 2.6 mm, 2.7 mm, 2.8 mm, 2.9 mm, 3.0 mm, 3.2 mm, 3.4 mm, 3.5 mm, 3.6 mm, 3.7 mm, 3.8 mm, 3.9 mm, or 4.0 mm or a value in the range between any two of the aforementioned values.

The sheet 200 is distortable at temperatures of between 50 and 70 deg Celsius, depending on the polycaprolactone content. Typically the sheet is brought into the malleable condition by heating in a water bath set around 5 degrees Celsius above the melting temperature, most preferable at 65 deg Celsius. It alternatively, be heated using a convection or fan oven, or a microwave oven. An impression of the face, particularly in the cheek region around the nose, is taken by placing the first 50 side i.e. skin contact or facing side 32 in contact with the skin and/or hair, and applying pressure to the sheet 200.

After molding, the sheet 200 is allowed to cool, hardening in the process. A resulting nose mask 10 is shown in FIGS. 2, 3, 15, 16, 18 and 19. It is noted that the shape of the mask 10 may be adjusted by the application of hot air to the exterior surface 30; this allows the shape to be fine-tuned, for instance, to relieve pressure points experienced by the wearer. Hot air may be provided by a hot-air blower (e.g. a hairdryer).

The sheet 200 exhibits excellent deformability properties, conforming to the shape of the face without the need to apply excessive pressure. The first layer 55 where present provides a comfortable wearing against the skin, and prevents adhesion to the skin and/or hair by the core layer 60. It also insulates the heat emitted by the heated core 60 layer from the skin of the subject. The second layer 65 where present prevents the core 60 from adhering to itself or other articles, and in addition reduces the hardening time. The sheet is considerably thinner than conventional masks, it is semi-rigid, has a soft touch feeling, and, therefore, provides a high degree of comfort in both molding and wearing.

The core layer 60 of a multilayer sheet material comprises a thermoplastic composition containing polycaprolactone and polyurethane. It has a thickness between 1 mm, 1.5 mm, 2.0 mm, 2.5 mm, 3 mm or a value in the range between any two of the aforementioned values, preferably between 1 mm 3 mm.

The polyurethane may present in an amount of 0%, 10%, 20%, 30%, 40% or 50% (w/w), or a value in the range between any two of the aforementioned values, preferably 20 to 40%, most preferably 30%. The polycaprolactone may be present in an amount of 60%, 70%, 80%, or 90% (w/w) or a value in the range between any two of the aforementioned values, preferably 60 to 80% (w/w), most preferably 70%. Typically, there will be more polycaprolactone than polyurethane which polycaprolactone lowers the temperature at which the sheet deforms. The ratio of polycaprolactone:polyurethane is (w:w) may be 5:1, 4:1, 3:2, 3:1, 2.3:1, 2:1 preferably 2.3:1.

The molecular weight of the polyurethane may be equal to or less than 10 000, 20 000, 30 000, 40 000, 50 000, 60 000, 70 000, 80 000, 90 000, 100 000, 120 000, 140 000, 150 000, or a value in the range between any two of the aforementioned values, preferably between 10 000 and 100 000. Polyester polyurethane is the preferred polyurethane.

The molecular weight of the polycaprolactone may be 10 000, 20 000, 30 000, 40 000, 50 000, 60 000, 70 000, 80 000, 100 000, 200 000, 300 000, 400 000, 500 000, or a value in the range between any two of the aforementioned values, preferably between 10 000 and 60 000, more preferably between 37 000 and 500 000.

Caprolactone polyester polyurethane is particularly suitable, which polyurethane may be obtained by reacting isocyanate and polycaprolactone-based polyester. Such a caprolactone polyester polyurethane is commercially available as a granulate. The melting point of said polycaprolactone polyester polyurethane lies between 190 and 210 degrees Celsius. By adding the polycaprolactone, also preferably in granulate form, a thermoplastic composition is obtained that is distortable and kneadable at a temperature of ˜69 degrees Celsius and remains distortable by cooling down about 50 degrees Celsius. At this temperature, the core layer 60 may be stretched at least up to twenty times the original length thereof. In the hardened condition, the thermoplastic composition is semi-rigid and has a memory effect that, after heating, returns to the shape formed on cooling. It is non-elastic in the hardened condition.

Advantageously, the core layer 60 comprises between 1 to 40% (w/w) of microspheres of non-metallic, heat-accumulating material which is especially suited for heating in a microwave oven. Preferred are glass microspheres with a diameter between 20 and 800 micrometer. A colouring agent may be added to the core.

One or more intervening layers 70, 70′ (FIG. 24) may be disposed between the core layer 60 and the first outer layer 55, and/or disposed between the core layer 60 and the second outer layer 65.

Typically an intervening layer 70, 70′ comprises a higher polycaprolactone content compared with the core layer 60, and thus can be deformed at a lower temperature than the core layer 60. According to one aspect of the invention, an intervening layer 70, 70′ has a polycaprolactone content that is higher than the core layer 60 by 5%, 10%, 15%, 20%, 25%, 30%, 50%, 100%, 200%, 300%, or 400% or a value in the range between any two of the aforementioned values, preferably between 100% and 400%. An intervening layer 70, 70′ may be comprised of pure polycaprolactone.

Thickness of an intervening layer 70, 70′ may be 20%, 30%, 40%, 50%, 60%, 70% or 80% that of the core layer 60 or a value in the range between any two of the aforementioned values, preferably between 40% and 60%.

By virtue of an intervening layer 70, 70′, the sheet 200 can be deformed at a lower temperature, since only the only the intervening layer 70, 70′ of the sheet 100 needs to be heated above the temperature at which the thermoplastic material deforms; the result is that heating up time before use is shorter and comfort to the subject is improved. As the core layer 60 has not to be brought at such high temperature, the expansion of the core 60 is also smaller. Due to expansion when heating, successive windings could be pressed so strongly together that they adhere strongly to each other.

In an advantageous embodiment of the invention, the intervening layer 70, 70′ comprises between 1 to 40% (w/w) of microspheres of non-metallic, heat-accumulating material which is especially suited for heating in a micro-wave oven. Preferred are glass microspheres with a diameter between 20 and 800 micrometer. A colouring agent may be added to the intervening layer 70, 70′.

The first outer layer 55, also referred to as the ‘first layer’ herein is a body-side liner i.e. the skin contact or facing layer. It may be made from any material that is inert and provides a non-irritating and comfortable feeling against the skin such as felt, flocking material, neoprene or a knitted material.

When the first outer layer 55 is neoprene, it may be neoprene or an elasticated neoprene.

It may be elasticated by virtue of a layer of elastic material on one side. The neoprene may be disposed with a plurality of perforations to increase breathability. The perforations particularly assist circulation when aeration apertures are limited to the layers of the thermoplastic material and do not extend through to the first outer layer 55 and optionally second outer layer 65. The perforations may have a diameter of 0.1 mm, 0.2 mm, 0.3 mm, 0.4 mm, 0.5 mm, 0.6 mm, 0.7 mm, 0.8 mm, 0.9 mm, or 1 mm or a value in the range between any two of the aforementioned values. Such perforations assist, for example, with heating the core layer 60 by providing access directly to the core, and with drainage when heating the sheet occurs in a warm water bath.

When the first outer layer 55 is a knitted material, it comprises a polyamide-based knitted fabric material with a thickness outside the core layer 60 of between 0.05 and 1.5 mm. According to one aspect of the invention, the knitted fabric material is formed from a yarn comprising between 80% to 95% polyamide, and between 5% and 15% elastane, preferably comprising 90% polyamide and 10% elastane. The fabric weight may be 190 g/m², 200 g/m², 210 g/m², 220 g/m², 230 g/m², or 240 g/m², preferably between 210 g/m² and 230 g/m². The fabric may be coloured, for example, with a calming colour such as a neutral tone, pastel shade, or primary colour such as blue.

When the first outer layer 55 is a flocking material, the material may be any finely cut natural or synthetic fiber. It is applied so as to produce a velvet finish. The flock may be coloured, for example, with a calming colour such as a neutral tone, pastel shade, or primary colour such as blue.

The thickness of the first outer layer 55 is equal to or less than 0.025 mm, 0.05 mm, 0.06 mm, 0.08 mm, 0.1 mm, 0.5 mm, 0.8 mm, 1 mm, 1.2 mm, 1.5 mm or a value in the range between two of the aforementioned values, preferably between 0.05 and 1.5 mm, more preferably having a thickness of between 0.1 and 0.4 mm.

The core 60 is bonded to the first layer 55 or intermediate 70, 70′ layer by virtue of the adhesive property of the core 60 in the fluid condition. Adhesion may be enhanced pressing the first layer 55 onto the core layer 60, for example, by the use of roller during manufacture.

Due to the presence of first layer 55, the sheet 200 may be applied directly in distortable condition on the skin. It does not adhere to hair and skin. It does not either leave visible fingerprints, so that disposable gloves not necessary for the application. In addition, the first layer 55 forms a thin insulating layer, such that the skin is not subject to elevated temperatures that might otherwise harm such areas.

The first layer 55 is the skin-contact surface of the mask 10 and thus provides a pleasant feeling against the skin, and also allows a flow of circulating air across (rather than through the mask in the absence of aeration apertures), thereby reducing the build up of perspiration and heat below the mask.

The second layer 65, also referred to as the ‘second layer’ herein, comprises a soft resilient open cell foam plastic disposed over the core layer 60 and forming contact therewith or with the optional intermediate layer 70, 70′.

The second layer 65 forms a physical and insulating layer which protects the core layer 60 in the malleable condition. The foam plastic outside the core layer 60 or optional intermediate layers 70, 70′, before the application, does not comprise thermoplastic material as for example the foam plastic from the composite material according to U.S. Pat. No. 3,728,206 which foam plastic does not form a coating. The second layer 65 forms a covering which prevents portions of the sheet 200 from adhering inadvertently to one another. The second layer 65 allows the sheet to be handled with bare hands without, damage to the core 60 or optional intermediate layers 70, 70′, and without adhering to the fingers.

The thickness of the second outer layer 65 is equal to or less than 0.05 mm, 0.06 mm, 0.08 mm, 0.1 mm, 0.5 mm, 0.8 mm, 1 mm, 1.2 mm, 1.5 mm or a value in the range between two of the aforementioned values, preferably between 0.05 and 1.5 mm, more preferably having a thickness of between 0.4 and 0.6 mm. It has been found that layers of foam plastic with a thickness outside of the core of more than 1.5 mm would prevent a good adherence to the underlying layer (i.e. core or intermediate layer) when the sheet is deformed.

The second layer 65 is made from a material able to withstand temperatures at which the core is softened or weakened. They are preferably made from a non-thermoplastic plastic, such as polyurethane formed into soft, open cell foam. The foam that has such an open-cell structure that core layer 60 in softened condition can traverse it when pressure is exerted but also that the second layer 65 can be elastically deformed without tearing. When the sheet 200 is heated, it can be stretched up to four times its original length without breakage of the second layer 65. Suitable foam plastics for the second layer 65 are polyurethane, particularly polyester polyurethane and polyether foam.

The core 60 is bonded to the second layer 65 or intermediate 70, 70′ layer by virtue of the adhesive property of the core 60. Adhesion may be enhanced pressing the second layer 65 onto the core layer 10, for example, by the use of roller during manufacture.

For some applications, the second layer 65 may be provided with perforations (not shown) with a diameter of at least 0.5 mm and for example 2 mm. Such perforations assist, for example, with heating the core layer 60 by providing access directly to the core, and with drainage when heating the sheet occurs in a warm water bath.

Cross-wise through core 60, first 55 and second 65 layers, smaller perforations with a diameter of at least 0.5 mm and preferably about 1 to 1.3 mm may be provided, so as not to hamper the skin breathing after applying the sheet. Said perforations may lie on rows crossing each other under 90 degrees and making an angle of 45 degrees with the transversal direction of the sheet of composite material, at a distance of each other in the rows of 1.5 to 4 mm. The skin may still breathe even after application of the material.

The support is provided with a pair of strap fixtures 14, 16, adapted to affix a strap that passes around the back of the head, and secures the mask 10 to the face of the subject. The fixtures 14, 16 for the strap may be a pair of slots as depicted, for instance, in FIGS. 1 to 3, 9D, 10D, 12, 14 to 19, however, it is not necessarily limited thereto. The fixtures 14, 16 may comprise one part of a Velcro strip, or one part of a press-stud or snap-fastener, which attaches to a reciprocating element on the strap. Each fixture 14, 16 may alternatively comprise a pair of holes as indicated, for instance, in FIG. 4. The strap is preferably made from an elasticated substance, such as a strip of elasticated material, and may include a length adjuster, to adjust the length of the strap and thus pressure exerted by the mask 10 on the face.

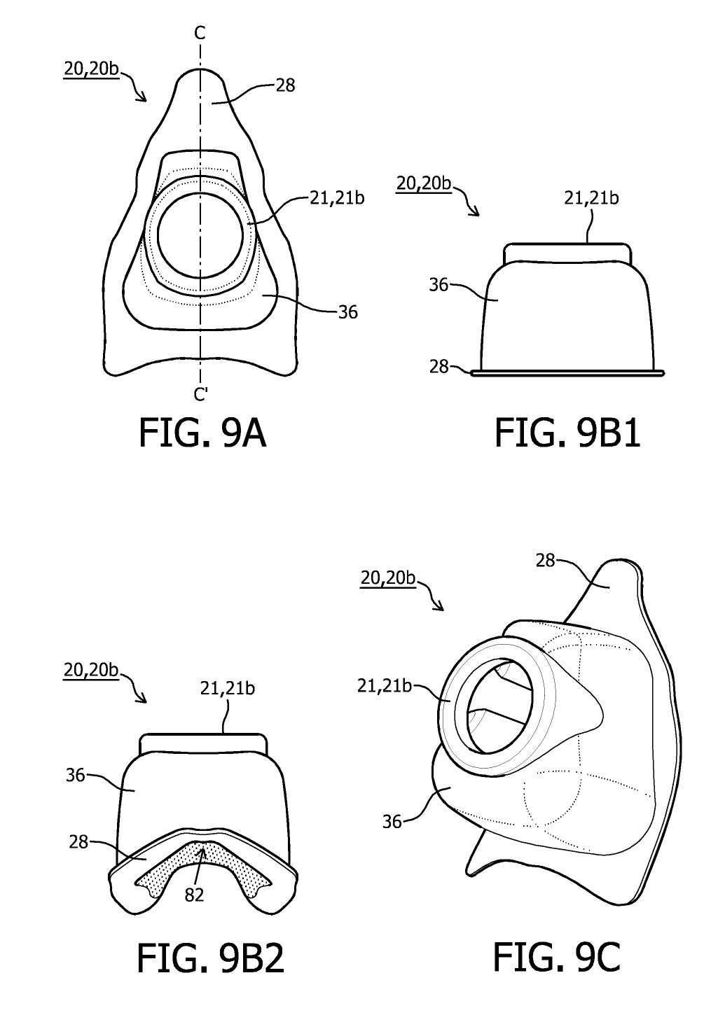

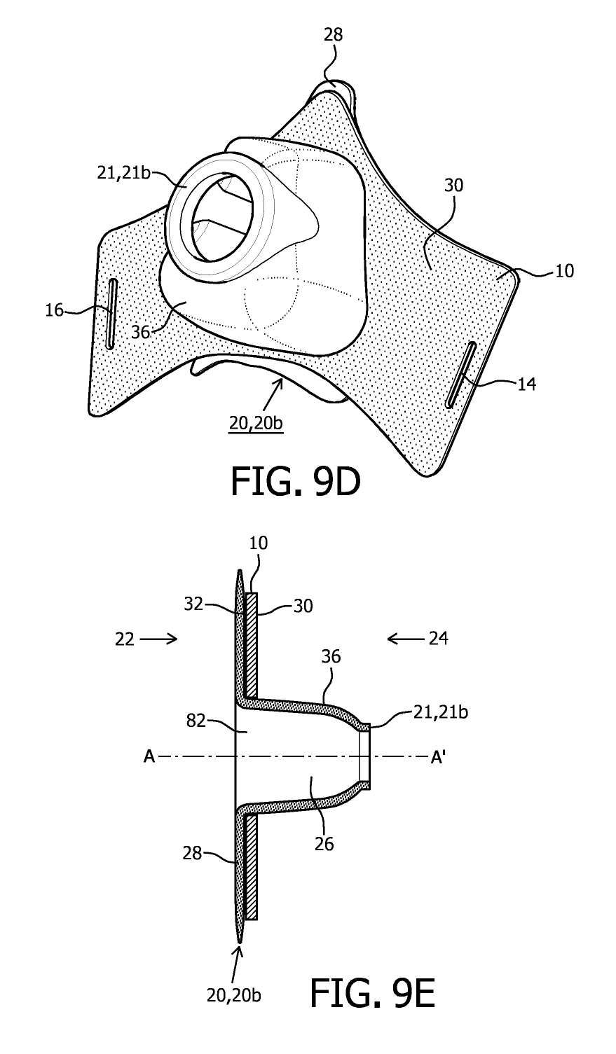

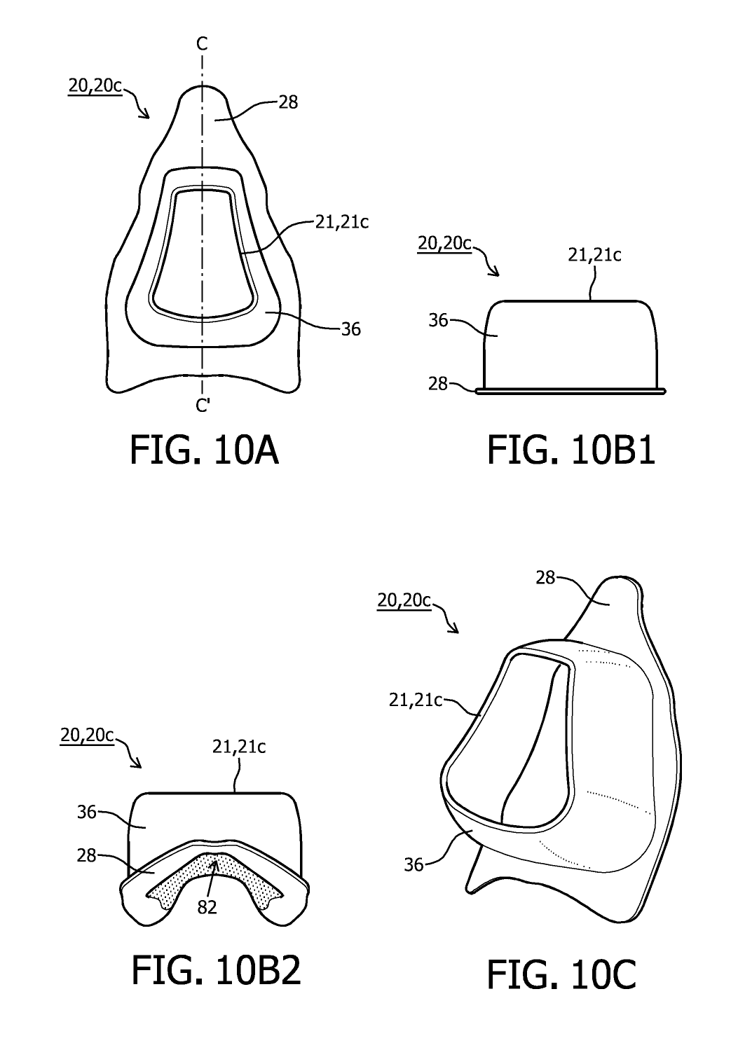

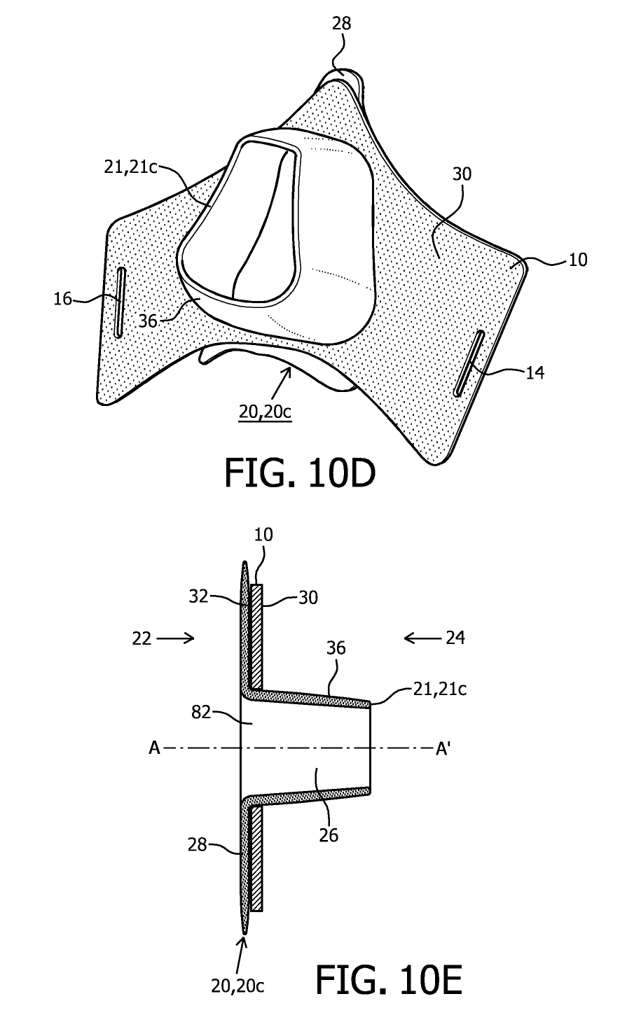

The support 100 also includes one or more couplings 20, 20a-c, 40, 45 for a tube such as a catheter for the delivery of fluid to the nose of the subject. The support 100 maintains the position of the tube relative to the face, permitting the subject to receive treatment for prolonged periods while being able to move the head freely. The support 100 prevents the tube from inadvertently disconnecting from the nose. The coupling 20, 20a-c, 40, 45 may be permanently or dismountably fixed (e.g. 20a-c) to the mask 10. According to one embodiment of the invention (FIGS. 1 to 9), the coupling is comprised in a hollow tubular fitting 20, 20a-c, provided at one (proximal) end with an opening 82 for receiving the nose, and at the other (distal) end with an inlet port 21 for coupling to a fitting for providing gas (e.g. air). The proximal end is in connection with the nose aperture 12 of the mask 10. The tubular fitting 20, 20a-d may be essentially longitudinal as shown in FIG. 4, having a longitudinal (central) axis A-A′ between the proximal 22 and distal 24 ends. It is provided with a passageway 26 suitable for the passage of fluids, that connects the inlet port 21 to the opening 82 at the proximal 22 end. As shown in FIG. 4, the port 21 may have an essentially triangular profile when viewed the along the A-A′ axis towards the proximal 22 end, making it suitable for attachment to existing triangular surgical couplings for standard nose masks. The triangular shape may be evident as an isosceles trapezium as shown, for example, in FIGS. 9C and 10C. According to one embodiment of the invention, the port 21 is adapted to receive a triangular profile gas coupling for a standard nose mask.

The terms “distal” and “proximal” are used through the specification, and are terms generally understood in the field to mean towards (proximal) or away (distal) from the wearer. Thus, “proximal” or “proximal side” means towards the wearer. Conversely, “distal” or “distal side” means away from the wearer.

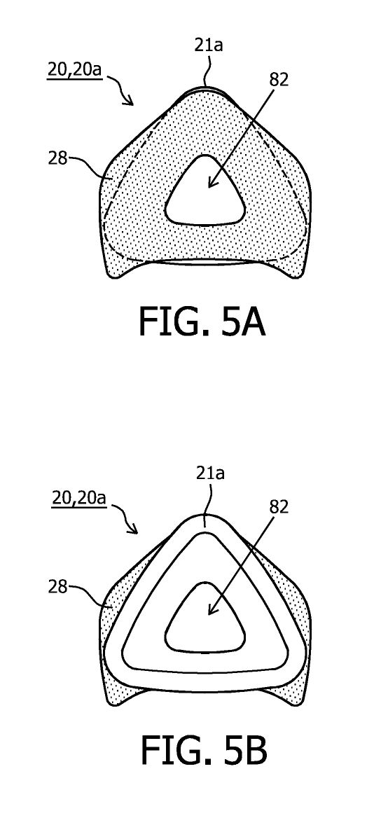

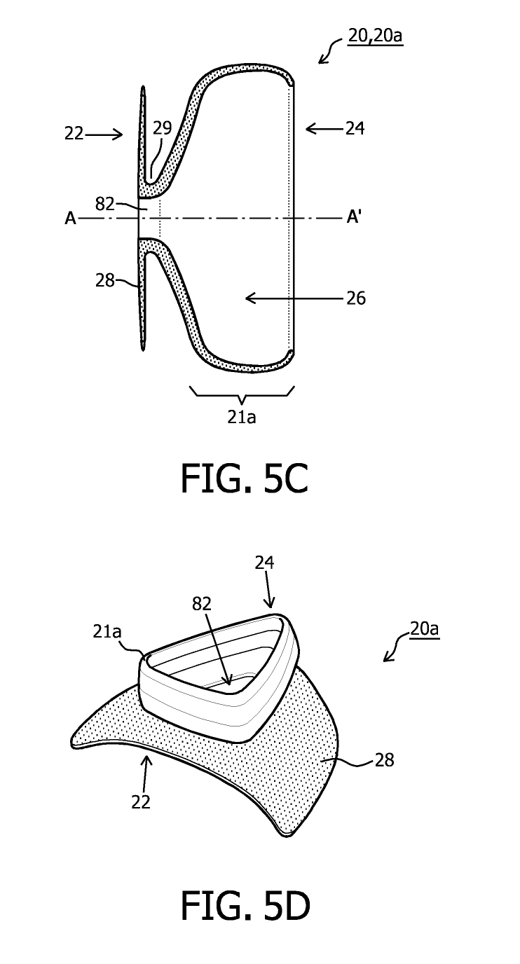

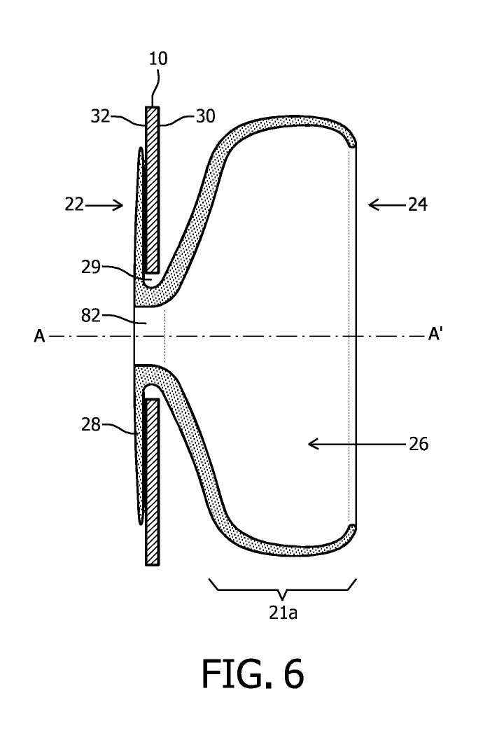

According to one embodiment of the invention, the coupling is a tubular fitting 20a configured for dismountable attachment to the nose mask 10. The dismountable attachment may be achieved by any means. Preferably, the proximal end 22 of the dismountable tubular fitting 20a is extended with an open, flexible flange 28, (FIGS. 5A, B, C, D) which flange 28 is configured to pass through the nose aperture 12 in the mask 10 in a folded condition such that the flange 28 can be disposed on the skin-facing side 32 of the nose mask 10 (FIG. 6), while the inlet port 21a disposed on the exterior side 30 of the mask 10, thereby dismountably attaching the removable coupling 20a to the nose mask 10.

More in particular, the tubular fitting 20a may be essentially longitudinal as shown in FIG. 5C, having a longitudinal (central) axis A-A′ between the proximal 22 and distal 24 ends. It is provided with a flange 28 around the opening 82 at the proximal 22 end, the inlet port 21a at the distal end 24, and a passageway 26 connecting the inlet port 21a to the flanged opening 82. The flange 28 has a proximal 22 (skin facing) side and a distal 24 (mask facing) side. The opening 82 is essentially central to the area of the flange, and adapted to receive the nose.

Attachment of the tubular fitting 20a provided with the flanged 28 opening 82 is effected by advancing the flexible flange 28 in a folded state through the nose aperture 12 of the nose mask 10. Having passed through the nose aperture 12 from the exterior side 30, the flexible flange 28 unfolds and abuts the skin-facing 32 side of the mask 10. The inlet port 21a is disposed on the exterior side 30 of the mask 10, so sandwiching or suspending the mask 10 between the flange 28 and the port 21a, thereby dismountably attaching the removable coupling 20a to the nose mask 10. As such, the flange 28 has an unfoldable property that allows folding for passage through the nose aperture 12, and unfolding once in situ abutting the skin-facing 32 side of the mask 10.

The flange 28, besides providing a gas-sealing effect against the skin, also gives a comfortable feeling, and, having a mechanical buffering property, can mitigate frictional abrasive or indent effects the rigid nose mask 10 may impose during wearing. The removable coupling 20a provided with the flange 28 can dismounted and replaced after regular intervals or after sufficient wear and tear, so reducing the build up of dust and allergens; the lifespan of the wearable support 100 is thereby prolonged. As the flange is flexible, its shape is adapted to the molded form of the nosemask 10 and it need not have any thermoplastic properties. It is anticipated that different sizes of flange 28 would be available to suit different sizes and shapes or nose mask.

The flange 28 of the dismountable coupling 20a forms the proximal 22 wall of a groove 29 configured to seat the inside edge of the nose aperture 12. The groove 29 is preferably ring-shaped. The groove interior is preferable aligned essentially perpendicular to central A-A′ axis of the fitting 20a. The groove 29 is disposed along the circumference of the flange 28. The flange 28 is preferably oriented essentially perpendicular to the longitudinal A-A′ axis of the dismountable tubular fitting 20a. The groove 29 preferably has a triangular profile when viewed along the A-A′ axis towards the distal 24 end. The groove 29 preferably has a triangular profile when viewed along the A-A′ axis towards the distal 24 end. The triangular profile of the groove 29 reciprocates the triangular profile of the nose mask 10 aperture 12. The triangular profile of the groove 29 may be smaller in size than that of the port 21a.

As mentioned above, the flange 28 of the dismountable coupling 20a forms the proximal 22 wall of a groove 29; the distal 24 (other) wall of the groove 29 may be formed from the back (proximal) wall of the port 21a as shown in FIG. 5C and 6.

As shown in FIG. 6 the flange 28 adjoining the groove 29 is adapted to flank the sub-region of thermoplastic material disposed around the nose mask 10 aperture 12 on the skin-facing 32 surface, thereby sealing the dismountable tubular fitting 20a against the nose mask 10.

The flange 28 is preferably formed from any suitable material exhibiting the requisite unfoldability property i.e. the ability to be folded and unfolded without weakening or other damage. Preferably, the coupling is formed from a flexible material such as silicone.

The flange 28 may have any profile when viewed along the A-A′ axis towards the distal 24 end, but in general, it will at least partially correspond with the shape of the nose mask 10. The profile refers to the outer shape, or the profile of the opening 82, or both. By correspond, it is meant that they essentially overlap, but some deviation is permitted, for example, the profile of the flange 28 may differ in area compared with the profile of the nose mask 10 by 0%, ±10%, ±15%, ±20%, ±25% or a value between any two of the aforementioned values.

While the profile of flange 28 at least partially corresponds with that of the nose mask 10 according to one embodiment, an independent description of the profile of the flange 28 now follows. The flange 28 is dimensioned to fit around the nose of the subject. It preferably extends from the nose around at least part of the cheeks. The flange 28 is disposed with an opening 82 for the nose that is preferably triangular in shape, but may alternatively be round, square, oblong or any other suitable shape. The shape will be largely determined by the shape of the nose. As will be appreciated, the flange 28 may be dimensioned so as not to extend over the eyes, mouth or ears. The flange 28 is devoid of apertures for the eyes, mouth or ears. However, an edge of the flange 28 may extend partially around, but not over, the eyes, mouth or ears.

The flange 28 may have a substantially longitudinal shape. With reference to FIG. 8, the flange 28 has a longitudinal (w) direction, and perpendicular thereto, a lateral direction (h). The longitudinal (w) direction is aligned with the base of the nose (i.e. parallel to the eyes). The opening 82 is preferably centrally located along the longitudinal length of the flange 28. The opening 82 may also be centrally located with respect to the lateral height. The flange 28 thus comprises two wings which extend longitudinally from and flank the opening 82. Each wing is adapted to extend from the nose around at least part of the cheeks.

The flange 28 may be planar or flat in the native state, as shown for instance in FIG. 9B1. According to an alternative embodiment, the flange may be curved in the native state as shown, for example, in FIG. 9B2. The curvature may be around the central lateral axis (C-C′). The curvature provides a closer fit of the flange against skin.

It is within the scope of the invention that the outer shape of the flange 28 is identical to that of the nose mask 10, or corresponds at least partially to that of the nose mask 10. The outer shape of the flange 28 may at least partially extend beyond the profile of the nose mask 10 or vice versa.

The dimension of the longitudinal width (WRL) of the flange 28 may be defined as the surface distance between the extremities of the opposing longitudinal edges of flange 28. The distance is measured in the longitudinal (w) direction. The dimensions of the longitudinal width (WRL) will depend on the profile of the nose mask 10, and may differ by 0%, ±10%, ±15%, ±20%, ±25% or more.

The dimension of the lateral height (HRL) of the flange 28 may be defined as the minimum surface distance between the extremities of the opposing lateral edges of the mask. The distance is measured along the lateral (h) direction. The dimensions of the lateral height

(HRL) will depend on the profile of the nose mask 10, and may differ by 0%, ±10%, ±15%, ±20%, ±25% or more.

The dimension of the longitudinal width (WRL) is larger than that of the lateral height (HRL) of the flange 28. Preferably the dimension of the longitudinal width (WRL) is 1.5, 2, 2.5, 3, 4, 5, or 6 times larger than that of the lateral height (HRL), or a value in the range between any two of the aforementioned values, preferably between 1.5 and 3 times.

The opening 82 for the nose is dimensioned such that the nose substantially passes there through. It is of sufficient size that at least the nasal tip passes entirely through the opening 82. The opening 82 may be triangular, round, oblong, square of any other suitable shape. It may have the shape substantially of an isosceles trapezium. The shape will be largely determined by the shape of the nose. Preferably, the opening 82 is substantially triangular in shape, the triangle having a base that corresponds to the nostril region of the nose. The triangle also has an apex that corresponds to the nasal bone. The orientation of the triangle is such that the base lies in the longitudinal direction (w) of the flange 28; the triangle base does not lie in the lateral direction (h) of the flange 28. Preferably the dimension of the base of the triangle (BRL) is 1 cm, 2 cm, 3 m or 4 cm in length. Preferably the perpendicular height of the triangle relative to the base (KRL) is 3 cm, 4 cm, or 5 cm in length. The profile of the opening 82 of the flange 28 may differ in area compared with the profile of the aperture 12 of the nose mask 10, by 0%, ±10%, ±15%, ±20%, ±25% or more.

The thickness of the flange 28 will generally be determined by the material used for construction, and by the requisite unfoldability property i.e. the ability to be folded and unfolded without damage. However, as a general guidance, the minimum thickness of the flange 28 will be equal to or less than 0.5 mm, 1 mm, 2 mm, 3 mm, 4 mm, or a value in the range between any two of the aforementioned values.

According to one embodiment of the invention, the coupling is a tubular fitting 20b exemplified in FIGS. 9A, 9B1, 9B2, 9C, 9D configured for dismountable attachment to the nose mask 10. The dismountable attachment may be achieved by any means. Preferably, the proximal end 22 of the dismountable tubular fitting 20b is provided with an open, flexible flange 28, at one end, and an inlet port 21, 21b at the other end (FIGS. 9A, 9B1, 9B2, 9C, 9D). The inlet port 21 may be circular 21b as shown in FIGS. 9A, 9B1, 9B2, 9C, 9D, and an elongated passageway 26 connects the inlet port 21b to the flanged opening 82. The outer wall 36 of the passageway 26 has an essentially triangular or essentially isosceles trapezium transverse profile the outer profile being the outer profile of a plane perpendicular to the A-A′ axis. The wall is intact and fluid impermeable to prevent leakage fluid. The inlet port 21b and outer wall 36 of the passageway are dimensioned to slidably pass through the nose aperture 12 in the mask 10 without substantial hinderance, such that the flange 28 is disposed on the skin-facing side 32 of the nose mask 10 (FIG. 9D, 9E), while the inlet port 21b and the substantial part of the passageway wall 36 are disposed on the exterior side 30 of the mask 10, thereby dismountably attaching the removable coupling 20b to the nose mask 10.

More in particular, the tubular fitting 20a may be essentially longitudinal as shown in FIG. 9E, having a longitudinal (central) axis A-A′ between the proximal 22 and distal 24 ends. It is provided with a flange 28 around the opening 82 at the proximal 22 end, the inlet port 21b at the distal end 24, and a walled 36 passageway 26 connecting the circular inlet port 21b to the flanged opening 82. The flange 28 has a proximal 22 (skin facing) side and a distal 24 (mask facing) side. The opening 82 is essentially central to the area of the flange, and adapted to receive the nose.

Attachment of the tubular fitting 20b provided with the flanged 28 opening 82 is effected by advancing the inlet port 21b and passageway 26 through the nose aperture 12 of the nose mask 10. The passageway is dimensioned to pass through the aperture essentially unhindered. Having passed the inlet port 21b and passageway 26 through the nose aperture 12 from the skin-facing 32, the distal side of the flexible flange 28 abuts the skin-facing 32 side of the mask 10. The inlet port 21b and substantially all the passageway 26 are disposed on the exterior side 30 of the mask 10, thereby dismountably attaching the removable coupling 20b to the nose mask 10.

The flange 28, besides providing a gas-sealing effect against the skin, also gives a comfortable feeling, and, having a mechanical buffering property, can mitigate frictional abrasive or indent effects the rigid nose mask 10 may impose during wearing. The removable coupling 20b provided with the flange 28 can dismounted and replaced after regular intervals or after sufficient wear and tear, so reducing the build up of dust and allergens; the lifespan of the wearable support 100 is thereby prolonged. As the flange is flexible, its shape is adapted to the molded form of the nosemask 10 and it need not have any thermoplastic properties. It is anticipated that different sizes of flange 28 would be available to suit different sizes and shapes or nose mask.

The flange 28 is preferably formed from any suitable material bio-compatible material that exhibits a sealing effect. Preferably, it is formed from a flexible material such as silicone. The wall 36 of the passageway 26 and the port 21 b are preferably formed from the same material.

The flange 28 may have any profile when viewed along the A-A′ axis towards the distal 24 end, but in general, it will at least partially correspond with the shape of the nose mask 10. The profile refers to the outer shape, or the profile of the opening 82, or both. By correspond, it is meant that they essentially overlap, but some deviation is permitted, for example, the profile of the flange 28 may differ in area compared with the profile of the nose mask 10 by 0%, ±10%, ±15%, ±20%, ±25% or a value between any two of the aforementioned values.

While the profile of flange 28 may at least partially corresponds with that of the nose mask 10 according to one embodiment, an independent description of the profile of the flange 28 now follows. The flange 28 is dimensioned to fit around the nose of the subject. It preferably extends from the nose around at least part of the cheeks. The flange 28 is disposed with an opening 82 for the nose that is preferably triangular in shape, but may alternatively be round, square, oblong or any other suitable shape. It may have the shape substantially of an isosceles trapezium. The shape may be largely determined by the shape of the nose. As will be appreciated, the flange 28 may be dimensioned so as not to extend over the eyes, mouth or ears. The flange 28 is devoid of apertures for the eyes, mouth or ears. However, an edge of the flange 28 may extend partially around, but not over, the eyes, mouth or ears.

The flange 28 may have a substantially longitudinal shape. With reference to FIG. 8, the flange 28 has a longitudinal (w) direction, and perpendicular thereto, a lateral direction (h). The longitudinal (w) direction is aligned with the base of the nose (i.e. parallel to the eyes). The length in the lateral direction may be greater than that in the longitudinal direction. The opening 82 is preferably centrally located along the longitudinal length of the flange 28. The opening 82 may also be centrally located with respect to the lateral height (h). The flange 28 thus comprises two flaps which extend longitudinally from and flank the opening 82, and two flaps which extend laterally from and flank the opening 82.

It is within the scope of the invention that the outer shape of the flange 28 is identical to that of the nose mask 10, or corresponds at least partially to that of the nose mask 10. The outer shape of the flange 28 may at least partially extend beyond the profile of the nose mask 10 or vice versa.

The dimension of the longitudinal width (WFO) of the flange 28 may be defined as the surface distance between the extremities of the opposing longitudinal edges of flange 28. The distance is measured in the longitudinal (w) direction. The dimensions of the longitudinal width (WFO) will depend on the profile of the nose mask 10, and may differ by 0%, ±10%, ±15%, ±20%, ±25% or more.

The dimension of the lateral height (HFO, FIG. 13) of the flange 28 may be defined as the minimum surface distance between the extremities of the opposing lateral edges of the mask. The distance is measured along the lateral (h) direction. The dimensions of the lateral height (HFO) will depend on the profile of the nose mask 10, and may differ by 0%, ±10%, ±15%, ±20%, ±25% or more.

The dimension of the longitudinal width (WFO, FIG. 13) is preferably smaller than that of the lateral height (HFO) of the flange 28. Preferably the dimension of the longitudinal width (WFO) is 1.5, 2, 2.5, 3, 4, 5, or 6 times smaller than that of the lateral height (HFO), or a value in the range between any two of the aforementioned values, preferably between 1.5 and 3 times.

The opening 82 for the nose is dimensioned such that the nose substantially or entirely passes there through. It is of sufficient size that at least the nasal tip passes entirely through the opening 82. Preferably, it is of sufficient size that the nose passes entirely through the opening 82. The opening 82 may be triangular, round, oblong, square of any other suitable shape. It may have the shape substantially of an isosceles trapezium. The shape will be largely determined by the shape of the nose. Preferably, the opening 82 is substantially isosceles trapezium in shape, the isosceles trapezium having a base that corresponds to the nostril region of the nose. The isosceles trapezium also has an upper part that corresponds to the nasal bone. The orientation of the isosceles trapezium is such that the base lies in the longitudinal direction (w) of the flange 28; the triangle base does not lie in the lateral direction (h) of the flange 28. Preferably the dimension of the base of the isosceles trapezium (WFI) is 1 cm, 2 cm, 3 m or 4 cm in length. Preferably the perpendicular height (HFI) of the isosceles trapezium relative to the base is 3 cm, 4 cm, or 5 cm in length. The profile of the opening 82 of the flange 28 may be smaller in area compared with the profile of the aperture 12 of the nose mask 10, by 0%, 2%, 4%, 6%, 8%, 10%, 15%, 20% or more.

The thickness of the flange 28 will generally be determined by the material used for construction. However, as a general guidance, the minimum thickness of the flange 28 will be equal to or less than 0.5 mm, 1 mm, 2 mm, 3 mm, 4 mm, or a value in the range between any two of the aforementioned values.

The length of the passageway 26 (from the flange 28 to the inlet port 21b) is sufficient to receive the length of the nose, from the cheek to tip. As a general guidance, it may be 1 cm, 2 cm, 3 cm, 4 cm, 5 cm, 6 cm, 7 cm or longer, or a value in the range between any two of the aforementioned values.

According to another embodiment of the invention, the tubular fitting is identical to those described above and in FIGS. 9A to 9E above, except the inlet port 21, 21c is not circular (21b) but is essentially triangular or has an essentially isosceles trapezium shape as shown in FIGS. 10A to 10E. The description above applies directly to the embodiments shown in FIGS. 10A to 10E except for the non-circular port.

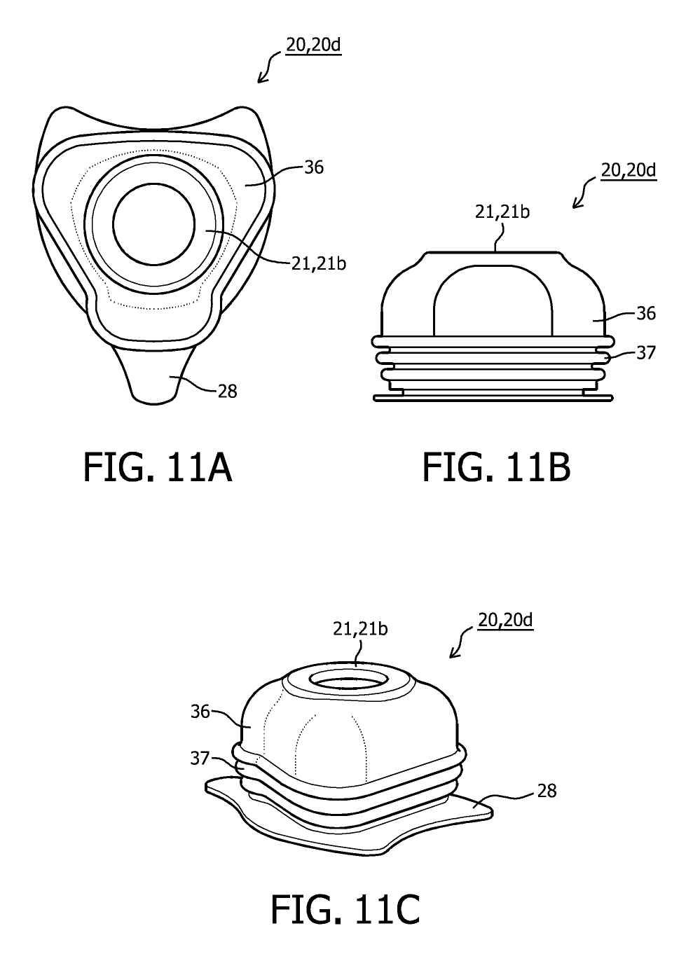

According to another embodiment of the invention, the tubular fitting 20, 20d is identical to those described above, and in FIGS. 9A to 9E and FIGS. 10A to 10E, except the wall of the passageway 36 is provided with a bellowed sub-region 37 as shown, for example, in FIGS. 11A to 11C. A bellowed region 37 is understood in the art, particularly in the application of bellowed tubing, and provides limited flexibility along the A-A′ axis. The bellowed region 37 allows the coupling 20d to bend and flex, thereby buffering tension applied to the coupling 20, 20d by way of the attached gas tube. The bellows may be in abutting alignment with the flange, as shown in FIGS. 11A to 11C. The bellowed region may be provided along 10%, 20%, 30%, 40%, 50%, 60% of the length of the passageway 26 or a value between any two of the aforemented values. The description above applies directly to the embodiments shown in FIGS. 11A to 11C except for the bellowed region.

According to another embodiment, the tubular fitting 20 is a triangular tube sealed over the nose aperture 12, i.e. sealed to the mask 10 exterior surface 30 using any suitable technique, such as employing adhesive, welding methods. The tube is permanently sealed over the nose aperture 12. The triangular tube is preferably made from a rubberized material, silicone, and is transparent.

According to another embodiment of the invention (FIGS. 13 to 15), the coupling is comprised in an adaptor 45 comprising a pair of adjustable outlet nozzles 46, 46′ for outlet of gas in fluid connection with an inlet port 48 for coupling to a fitting for providing gas (e.g. air). The outlet nozzles 46, 46′ are positioned on the mask for entry into the nostrils. Each nozzle body is essentially parallel to the central lateral axis (A-A′). The adapter 45 may be attached to the mask 10 exterior surface 30 using any suitable technique, such as employing adhesive, or welding methods. As shown in FIGS. 13 to 15, the nozzles 46, 46′ are arranged in alignment with the base of the triangular aperture 12 which alignment orients the nozzles 46, 46′ for insertion into the nose after molding. An adapter-type coupling 45 having the above described configuration is known in the art, for instance, as a nasal pillow. Examples of manufacturers of nasal pillows include ADAM and Resmed. Typically, they have a substantially polypropylene or polycarbonate body.

According to another embodiment of the invention (FIGS. 16 to 18), the coupling is comprised in a tube clip 40 configured to couple securely to a fluid delivery tube, such as a catheter, to the nose mask and align it with the entrance to a nostril. In FIGS. 16 and 17, the tube clip 40 is visible and is positioned below the base of the triangular nose aperture 12. The tube clip 40 may be any suitable for securing a catheter. The number of tube clips may be 1, 2, 3, 4, 5, 6 or more. Each tube clip may be adapted to secure 1, 2, 3, 4, 5, 6 or more tubes. The tube clip is preferably formed from a non-thermoplastic material such as polycarbonate or polypropylene.

1. A wearable medical support for delivery of fluids to the nose of a subject comprising:
a heat moldable nose mask, having a longitudinal direction, formed from a sheet of thermoplastic material configured for extending and for individual molding across at least part of the cheek bones of the subject, comprising:
a nose aperture dimensioned to fit the nose of the subject, and
a fixture for a strap at each opposing longitudinal end of the mask, and
a coupling dismountably fixed to the mask for attachment of one or more tubes for delivery of the fluid to the nose.

2. Support according to claim 1, where the coupling is comprised in a hollow tubular fitting having a proximal and distal end,
the proximal end of the tubular fitting being provided with a flanged opening for receiving the nose,
the distal end being provided with the inlet port for attachment to a tube for delivery of the fluid to the nose, and
a passageway connecting the inlet port to the flanged opening.

3. Support according to claim 2, wherein the flange is provided on a skin-facing side of the nose mask, the inlet port is provided on an exterior side of the nose mask, and the passageway is disposed through the nose aperture, so mounting the mask over the passageway, and in abutting alignment with the proximal side of the flange.

4. Support according to claim 2, wherein the inlet port and passageway are configured to pass slidably through the nose aperture.

5. Support according to claim 2, wherein the passageway is at least partially bellowed.

6. Support according to claim 1, wherein the nose aperture is essentially triangular or isosceles trapezoidal, the base of the triangle or isosceles trapezium oriented in the longitudinal direction of the nose mask.

7. Support, according to claim 1, wherein the longitudinal width (W) of the mask is between 16 cm and 22 cm.

8. Support, according to claim 1, wherein the longitudinal width (W) of the mask is larger than that of the lateral height (H) of the mask, preferably between 1.5 and 3 times larger.

9. Support, according to claim 1, wherein the nose mask comprises a thermoplastic composition containing polycaprolactone and polyurethane.

10. Support, according to claim 1, wherein the nose mask comprises is disposed with a plurality of aeration apertures.

11. Support, according to claim 10, wherein the aeration apertures are provided at a density of between 0.5 and 5 apertures per cm².

12. Support, according claim 9, wherein the sheet of thermoplastic material comprises a core layer, and wherein the core layer comprises 20% to 40%, polyurethane, and 60% to 80% (w/w) polycaprolactone.

13. Support, according to claim 2, wherein the flange is formed from silicone.

14. Support, according to claim 1, wherein the overall thickness of the thermoplastic sheet is between 1.5 and 2 mm.

15. Support according to claim 2, wherein the flange of the dismountable coupling as defined in has a profile corresponding at least partially to that of the nose mask.
